(19) United States
(12) Patent Application Publication
(10) Pub. No.: US 2026/0269086 A1
(43) Pub. Date: Sep. 10, 2026

(54) Follow Me: Method and System for Facilitating Communication Based on Sober Living Interests and Living a Sober Lifestyle

(71) Applicant: Stan Riley, Tyler, TX (US)

(72) Inventor: Stan Riley, Tyler, TX (US)

(21) Appl. No.: 19/072,301

(22) Filed: Mar. 6, 2025

(51) Int. Cl. G16H 80/00; G06Q 50/00; G16H 40/20; H04L 9/40

(52) U.S. Cl. CPC G16H 80/00; G06Q 10/40; G16H 40/20; H04L 63/0421

(57) ABSTRACT

Disclosed herein is method for sober based social networking comprising the steps of receiving, from a user, a login information into a network platform using a personal computing device, wherein the network platform is configured to select automatically layouts fit to accommodate different screen resolutions and applications designed to work specifically on different computing devices, registering the user on the network platform in a full user mode and an anonymous mode to provide the user an option to choose from the login modes and alternate between them, receiving a query from the registered user regarding rehabilitation, generating a list of medical practitioners registered with the network based on a processing of the query, and creating communication links with the one or more registered medical practitioners based on a selection by the registered user.

### BACKGROUND OF THE INVENTION

This invention relates to communication technology. In particular, the present invention relates to a method and system for facilitating communication on a social network between users based on sober living interests that supports and encourages living a sober lifestyle.

Humans inherently desire to communicate and connect with each other. Many technologies throughout history have provided a means for humans to communicate regardless of the physical location of the parties. Telegraphs, telephones, cell phones and computers all provide an easy to use communication platform but the introduction of the internet, personal computers, and smart phones has exponentially increased the ability of people across the planet to communicate and connect.

The internet expands communications to people or persons who prior to the internet may have had serious obstacles in connecting with likeminded people. People struggling with addiction need a means to communicate and connect with other addicts and recovering addicts aiming to live a sober lifestyle. Addiction recovery success rates increase drastically when an addict connects and communicates with people with similar recovery goals and lifestyle choices. However, addiction issues with drugs and alcohol can be a source of shame on the addict that can trickle down onto the addict's family causing some addicts to remain silent in order to protect their family name. Prior to the internet, the only way an addict could connect with other addicts was through in person meetings the addict physically attended. The invention of the internet and social media alleviated this unnecessary shame by allowing private communication on a personal level about addiction and recovery in an online setting without having to physically attend a meeting.

In general, current sober living resources are structured to handle issues or problems solely for addicts only rather than the entire population in general. It would therefore be desirable to have a support system or communication network that provides a means to allow an addict to connect online with other sober minded individuals without being limited to connecting through a communication resource that only connects addicts or requires the user to identify themselves as an addict in order to access the resources available. Fostering communication solely concentrated on sober living lifestyle choices allows people of all kinds, addicts and non-addicts, to connect and form bonds centered around living a sober lifestyle.

While social media fosters communication across all ages, races, backgrounds, and political parties comprising addicts and non-addicts, social networks such as Facebook, Twitter, and Instagram engage millions of users on limitless topics and unbounded connections. Although Facebook connects a massive amount of users, these connections are often surface based and lack any meaningful life altering involvements. In order to have meaningful life changing connections with other users, users need to connect on a deeper level of personal issues that directly affects the user's quality of life or addresses real problems the user is facing. Addiction to alcohol and drugs is a serious issue that connects addicts and sober minded people alike but not frequently discussed in any meaningful way on current social media sites. There is currently a need for people struggling with drugs and alcohol to connect with sober friends on social media and learn how to enjoy their hobbies and activities again in the framework of a sober lifestyle in order to regain a new normal fulfilling life.

When a person chooses to live a sober lifestyle, exposure to certain advertising campaigns, negative people, and experiences, can trigger an addict into relapsing. Seeing an ad while scrolling through social media for alcohol could be the image that deflects the addict off the sober path to engage in alcohol or drug use. Seeing an old friend while scroll through social media that the addict used drugs with in the past could trigger the person to relapse. These are just two examples of how social media could negatively affect a sober person struggling to remain sober. Social media for the masses is not structured to encourage a sober lifestyle and manage the triggers that could set off a negative chain reaction. The sober user is merely trying to connect with friends on social media, but instead is triggered to partake in drugs or alcohol by logging in to the current options of social media and viewing something that causes the person to stumble.

Moreover, Facebook users are becoming increasingly unhappy with the social network. User data of millions of users was shared or leaked without consent for political purposes. Personal user data of millions of users was also breached and/or sold for advertising purposes. The essence of Facebook of connecting people all over the globe has been tainted by some of these breaches causing people to delete their Facebook account altogether. To avoid these political pitfalls, a social network free from the network endorsing political posts, selling user data, and allowing data breaches is needed.

A social network focused on sober living that supports living a sober lifestyle has not ensued despite the desperate need. A social network that makes sober content and resources readily available to support the sober user but also protects the user from potentially harmful advertisements that could cause the user to relapse would solve a great need. The relaxed screening process for advertisements on popular social media outlets causes businesses, social groups, and school districts to use social media platforms that contain advertisements for products that do not align with their mission or goals. The lack of such a sober social network causes some addicts to avoid popular social media outlets in general.

There is a need for a method and system of online communication for finding and connecting individuals with addiction issues personally or within their family or friend group in a simple, fast, inexpensive, anonymous manner anywhere in the world that encourages and supports a sober living lifestyle. Additionally, there is a need for a method and system of online communication for finding and connecting individuals without addiction issues through online communications that choose to live and support others in living a clean and sober lifestyle.

Further, there is a need for a method and system of online communication for a communication platform that combines addicts and non-addicts, anonymously or as identifiable users based on user preference, that share a focus on living a clean and sober lifestyle as a choice. There is a need for a method and system of online communication solely focused on sober living lifestyle choices that allows people of all kinds, addicts and non-addicts, to connect and encourage a sober living lifestyle. Accordingly, there is a need for a method to provide a web portal that creates and displays web pages that allows the user to connect to social networking for sobering living lifestyle choice as well as allow for targeted advertising.

There is a need for a method and system of online network communication that connects sober minded people with sober events and activities that provides the sober person an alcohol and drug free way to attend social events with other sober friends committed to their sobriety.

### SUMMARY OF THE INVENTION

This invention has several features; no single one of them is solely responsible for its desirable attributes. By no means limiting the scope of the invention as expressed by the information that follows, the inventions more prominent features will now be discussed briefly.

This system offers addicts a social network and communication means that supports and encourages sober living as a lifestyle choice. This system also offers non-addicts a social network that supports and encourages sober living as a lifestyle choice and not solely a communication means for recovering addicts. Currently sobriety based resources to date are almost exclusively designed for recovering addicts alone and do not embrace non-addicts that are also interested in sober living.

The present disclosed system would provide addicts and non-addicts equal opportunities to communicate, coordinate, facilitate, and participate in sober living lifestyle choices and events. Communication between an addict with a non-addict broadens the scope of interaction among individuals with a similar lifestyle choice of a commitment to sobriety in ways that do not currently exist.

This system would additionally provide a way for family members and friends to participate alongside a known addict to support their recovery efforts. The network described herein provides a preventative and supportive sober living social network system. These and other advantages of the current invention, in addition to other inventive features, will be apparent from the description of the invention provided herein.

This invention's system and method combines addicts and non-addicts, anonymously or as identifiable users, based on user preference, that share a focus on living a clean and sober lifestyle, which connects people across all addiction and social spectrums that share a commitment to living sober lifestyle. This invention's system and method creates an online community of sober living individuals that share a commitment to sober living. This invention provides sober individuals fellowship with others with a similar sober lifestyle because of sharing sober attitudes, interests, and goals among users. A sober lifestyle based on a social networking platform provides a forum for sharing sober lifestyle choices with like-minded individuals.

The invention meets the foregoing need and provides a method for creating an online community that creates and displays sober living social networking in a safe environment. The present invention provides a method for developing a sober living webpage with advertising, comprising the steps of accumulating a sober living based social network, determining an advertising section based advertisements that encourage and support sober living choices, wherein a portion of the web page comprises at least an advertising section based on sober living lifestyle choices and wherein a section portion of the web page comprises at least one of chat sections, a contact section providing user information, a discussions section, an events calendar and an announcement section, and a third section comprises a new feed section, a sponsor section, and/or a gallery section.

The method may be accomplished such that the advertising section is at least one of an advertising banner, a video recording, and an audio recording. The user section may display name, picture of user, and location of user. The user may be an individual user or an entity. The news feed section may contain information related to a specific entity or location of event based on the user's location. The method may also comprise the step of including a sponsor portion configured for advertising specific to the user or event. The step of accumulating the database of users is accomplished by accepting user input of personal or entity information. The method may also comprise the step of a user identifying as a member of an organization or entity for access to a web page only allowed entry or use by its specific members.

Another exemplary embodiment of the present invention provides a method of creating a sober living social network page comprising: a registered user for network access, accumulating a database of information from the user, and preparing the web page based on information received from the accumulation of the community database information from the user, wherein the webpage generated encourages a sober lifestyle and encourages sober lifestyle choices. The web page may include an advertising section based upon the user data profile information from the user. The method may also comprise granting access to the internet social network web page for the user with the activities profile to individuals defined by the user.

The present disclosure illustrates various techniques and configurations to enable a user to view and present content on a social network based on sober living for individual and multiple users. This content is selected to be relevant to, or at minimum not discouraging, sober living choices of the users. The interaction systems descried herein enable a user to view and provide appropriate content to further the goal of sober living. The integration of user databases and user communication described herein further may facilitate communications using social networks and portable communication mediums to provide additional availability and delivery of sober lifestyle choices content in a variety of settings.

The present disclosure illustrates various techniques and configurations to enable a user or group to include and share events on the social network, such as a sober holiday party, and any person, group or entity that desires to participate in this sober interest event. The concept of connecting people, groups, and entities on a social network that could be formed around an event is encompassed in the present disclosure.

After reviewing the summary and detailed description, one will understand how the features of this invention provide its benefits, which include providing a system and method of facilitation communication based on user's desire to live a sober lifestyle. The method includes receiving, using a communication device, wherein the present invention is a communication method and system that provides addicts and non-addicts a communication platform and resource to communicate, coordinate, facilitate, and participate in sober living lifestyle choices.

The embodiments of the invention and the various features and advantageous details thereof are detailed more fully with reference to the non-limiting embodiments and examples that are described or illustrated in the accompanying drawings and explained in the following description. Embodiments set forth in the claims encompass all available equivalents of those claims.

### BRIEF DESCRIPTION OF THE DRAWINGS

FIG. 1 illustrates an example network environment associated with a social networking system.

FIG. 2 illustrates an example method for user sharing and viewing content in an example social-network environment.

FIG. 3 illustrates an example network page on a mobile device for user sharing and viewing content in an example social-network environment

FIG. 4 illustrates an example sharing of sober content.

FIG. 5 is illustrates an example social graph.

### DETAILED DESCRIPTION

The entire network platform is either directly or indirectly empowering and encouraging a sober lifestyle for users, members, and groups. Comments and posts may be on any variety of topics as long as they do not promote using or abusing drugs or alcohol. Simple posts about a member's daily life that does not specifically mention being sober are allowable and encouraged. Posts about a member's alcohol use or drug abuse are not allowed and will be deleted by the network's automatic filtering system, by reporting the violation from another member to network administration, or by the network administration itself. This example filtration and quality control system is not an exclusive list of the ways to control and filter the site's content but provided as an example of how the site's content will be monitored and purged of content not allowed on the site.

The overall goal and purpose of the site is to connect and empower users by providing a happy, healthy, resource for sober minded individuals that promotes fellowship among other sober individuals in a social media network. This sober social media network connects individuals within the online community through the network allowing users, members and groups to connect online to form a sober community. After the user, members and or groups connect online, they can receive, view, chat, and post comments about sober content and about living a sober lifestyle.

The user may receive and view sober content that members, groups, or advertisers may share. Similar to other social media website networks, the user has the freedom to post as frequently or infrequently as the user desires. The user may log in to view the content as frequently or infrequently as the user desires. The user may connect with as many members or as few of members as the user desires.

The user may have two log in modes: regular mode and anonymous mode. The user controls his privacy setting and can alternate between regular log in mode and anonymous mode. The user controls his own privacy and selects the amount of private information the user desires to share. The level of privacy selected may affect the amount of content accessible by the user. To overcome any accessibility issues, the user may adjust his privacy settings if necessary to access all desired content. If the user reviews a post about sober content from a group that interests the user, then the user can connect to the group by viewing the group's data information page.

When the user views a group's personal information page that he is interested in, he can view the sober content the group previously posted and shared. The user can also join local, non-local, or nationwide groups to further encourage a sober lifestyle if they find the content posted by the group interesting. The user can also see the list of members, location of the group, resources the group provides or endorses, or whatever information the group chooses to share on the site. The group may also have a private page the user must apply for or be approved to join. The group itself and not the network controls the members of their group page and sets the parameters for membership to their personalized site.

These groups or individual users/members can host sober events locally and advertise these events on their group page on the social network. The network may also be set up to house an events page where groups, individual users, or entities may share their upcoming event with the network. The groups or individual users can also host sober events and also advertise them on an event page. This event page functions like a calendar where users can view sober events by date. The user may filter the calendar to see upcoming events by date and location to view upcoming events within a certain mile radius to the user. Alternatively, the user can view all events regardless of location.

The network connects a user online with a community of sober members and sober groups, which ideally leads the user to participate in person in sober events with sober groups and sober members the user learns about on either the group's sober events page. Connecting sober user to sober members and sober groups both online and in person designed and configured by the present invention.

Sober events can be a group of sober individuals who all live in an urban city going to a sports event as a group who are committed to remaining sober. While there probably will be alcohol served at the sports event, the sober user and group attends the event without partaking in the alcohol. The sober user is able and encouraged to life a full, abundant, active social life with other sober like-minded individuals through the connections facilitated on the sober social network of the present invention.

Without a group of like-minded sober members, the user would not feel comfortable going to a sporting event where alcohol is being served for fear of being pressured to drink or not having the support required not to drink. The user would previously avoid the sporting event because of the temptation of alcohol at the event resulting in isolation from activities the user enjoys without the community of sober network participants the user connected with on the sober network. Connecting people online and by organized events, the present invention expands the user's social life and lifestyle choices to facilitate getting the user back to enjoying an active social life.

A majority of addicts recently out of rehab struggle to find a new place in society as a sober individual. Their past life and old friends often are not good influences or supportive of the former addicts new sober lifestyle. In order for the addict to remain sober, the addict needs to find a new group of friends with new sober friendly activities. Traditional meetings focused on sobriety are often filled with all ages, races, and socioeconomic status making it difficult for younger addict to connect and find other sober minded friends his own age, which is critical to staying sober.

In one embodiment, the average college aged adult connects with friends over social media. However, most of his friends are not sober and may have contributed to his past addictions. Once the college aged adult commits to sobriety, the college aged adult needs to connect with other sober college aged adults to join him on his walk to continue his sobriety. Using other popular social media sites to connect with other college aged adults more than likely will not further or encourage his sobriety.

Popular social media sites content frequently contains member posts and group pages that promote alcohol and drug abuse along with advertisers'ads promoting the services as well. In the present invention, network members'content and individual data must promote and exhibit a sober lifestyle. Pictures, posts, content, and events that do not promote a sober lifestyle are not allowed on the site. A frequently used method of screening, reporting, and removal of offensive content monitors the posts and removes offensive posts, users, advertisements or comments as needed. Any method currently used in the art to screen and clean up the site is effective. A reasonable standard will be in effect.

In the same way, advertisements of alcohol, marijuana, synthetic substances, drug paraphernalia, or controlled substance laced edibles are all frequently found on popular social media sites. Any one of these could be detrimental to the sober user and member's sobriety. Advertisements promoting substance use or abuse are not allowed on the present invention's network site. Preventing the user, member, and group from viewing these potential relapse causing advertisements protects the user from potential relapse triggers. All advertisements on the entire site should foster a sober lifestyle in a reasonable manner.

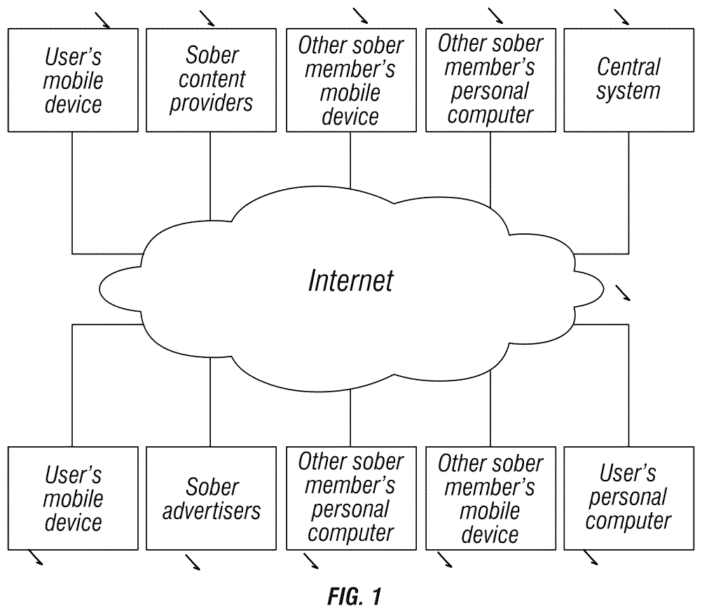

Applicants disclose, as illustrated in FIG. 1, an example network environment associated with a social-networking system 100 accessed by users 101, 106, 110 via the internet. Network environment includes connection to the social network 100, a user 101, 106, 110 a central system 105, sober content providers 102, and other sober network members 103, 104, 108, 109 connected to each other through the internet 100. Although FIG. 1 illustrates a particular arrangement of the social network 100, a user 101, 110, a central system 105, sober content providers 102, and other sober network members 103, 104, 108, 109 connected to each other through the social network 100, this disclosure contemplates any suitable arrangement of a user 101, 110, a central system 105, sober content providers 102, and other sober network members 103, 104, 108, 109 connected to each other through the social network via the internet 100.

As an example and not by way of limitation, a user 101, 106, 110, may be connected to the internet based social network 100 through the user's mobile device 101, user's second mobile device 106, and/or user's personal computer 110. The user 101, 106, 110 may be connected through one device or all three devices to the internet 100 at any time. Moreover, although FIG. 1 illustrates a particular number of user's devices (mobile device 101, user's second mobile device 106, or user's personal computer 110), this disclosure contemplates any number of user's devices 101, 106, 110 capable of connecting to the internet. As an example and not by way of limitation, network environment may include a user 101, 106, 110, a central system 105, along with any number of sober content providers 102, and/or any number of other sober network members 103, 104, 108, 109, connected to each other through the internet 100.

As another example and not by way of limitation, network environment may include a user 101, 110, a central system 105, along with any number of sober content providers 102, any number of other sober network members 103, 104, 108, 109, and any number of sober advertisers 107 connected to each other through the social network 100. At minimum, the network environment may include at least one user 101, 110, a central system 105, along with at least one sober content providers 102, at least one sober network members 103, 104, 108, 109, and at least one sober advertisers connected to each other through the social network 100.

In example embodiments, user 101, 106, 110 may be an individual (human user), a group (support group, entity, or collaboration of entities), or an entity that interacts, communicates or connects over a social network system 100. In example embodiments, member 103, 104, 108, 109 may be a group of users that communicate or connect over a social network system 100. In this example embodiment, the social network 100 may be a network based on a computing system hosting an online social network. Social network system 100 in the particular embodiment may create, store, receive, send, and filter social network data, such as example only, user profile information 101, 106, 110 or other suitable user data or sober profile information related to the online social network 100. Social network system 100 may be accessed by the other components 102, 103, 104, 107, 108, 109 of the network either directly or via network. In this particular embodiment, social networking system 100 may include an authorization server that allows user 101, 106, 110 and members 103, 106, 110 to opt in or opt out of having their actions logged by social networking system 100 or shared with other systems by setting appropriate privacy settings. User 101, 106, 110 may determine to not share any information with other members 103, 104, 108, 109 or systems to remain completely anonymous except for user's first name 101, 106, 110. User 101, 106, 110 and members 103, 104, 108, 109 may access social network system 100 by any suitable computing device, such as, for example, a personal computer, a laptop computer, a cellular telephone, a smartphone, or a tablet computer.

This example envisions any appropriate network 100. This network may include an ad hoc network, intranet, extranet, local area network, virtual private network, wireless local area network, wide area network, a wireless wide area network, a portion of the Internet, a cellular telephone network, or a combination of two or more of any of these.

All components of the social network 100 center on a sober lifestyle and all facets thereof. A plurality of members 103, 104, 108, 109 interested in sober lifestyle choices may join the social network 100 and form a community of members 103, 104, 108, 109 around the user 101, 106, 110. After a user 101, 106, 110 registers with the social network 100, the user 101, 106, 110 has access to sober content on the network 100; the user 101, 106, 110 can share sober content, and can view other member's 103, 104, 108, 109 sharable data and content. Advertisers 107 on the social network 100 and their advertisements are prescreened and only advertisers 107 that display content that promotes a sober lifestyle are allowed to advertise on the network 100. Advertisements 107 geared toward alcohol, involve alcohol use, suggest alcohol use, discuss or glamorize alcohol use or abuse are not approved on the network 100 and will be blocked by the network administration should an inappropriate advertisement be initially approved for the network 100. Additionally, any advertisements 107 of political nature are not approved on the network 100 and will be removed by network administration. User 101, 106, 110 and members 103, 104, 108, 109 may make comments or political posts on their own private pages, but paid advertisements on the network's main page with a political posture will be blocked.

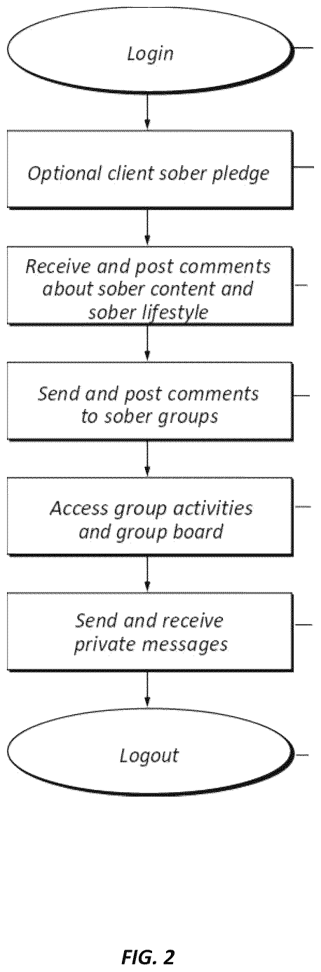

FIG. 2 illustrates an example method of single user 101, 106, 110 sharing and viewing content in an example social-network environment. In order to access the social network 100, the user 101, 106, 110 is required to log in to the network 100 via the user's suitable computing device. User 101, 106, 110 may access social network system 100 by any suitable computing device, such as, for example, a personal computer, a laptop computer, a cellular telephone, a smartphone, or a tablet computer.

The layout and design of the social network 100 may have minor differences due to formatting and screen resolutions to maximize user, member, advertiser, and group experience. For example, the social network may have a website design 110, a mobile website design 106, tablet design 101, and apps designed to work on specifically on each of these computing devices. While the formatting may be different, the spirit and sober lifestyle focus on content and data remains the same across all computing devices.

The login 200 page requires the user 101, 106, 110 to identify by username and password. First time use of the page allows the user 101, 106, 110 to select the username and password along with other identifiable private information to reach the social network 100 for all future login 200 access. If the user 101, 106, 110 desires to access the network 100 but does not want to be identified or identifiable, the user 101, 106, 110 can select anonymous mode as their login which allows the user 101, 106, 110 to only divulge the user's 101, 106, 110 first name to access the network 100. If the user 101, 106, 110 chooses anonymous mode, the user 101, 106, 110 may have limited access to certain features because of the limitations imposed by other members and groups 203, 204, 205 privacy settings in using the network in anonymous mode. In order to access the full spectrum of the network 100, the user 101, 106, 110 must login in the active user 101, 106, 110 mode.

The user 101, 106, 110 may divulge personal information about themselves to be displayed on the user's 101, 106, 110 personal information page on the network. The user 101, 106, 110 decides the amount of information to share and post on their personal information page. The user 101, 106, 110 is in control of their privacy and can choose how much of their personal information is displayed. The anonymous mode login option only displays the user's 101, 106, 110 first name as a way to closely replicate more traditional alcohol and drug recovery meeting formats.

In popular recovery support groups, participants only provide their first name as a way to protect their privacy and the optional log in mode for this network mirrors that privacy preference. Other social media networks require first and last name to access the features of their networks, which limits access to those individuals who prefer to keep their true identities private. The present invention allows the sober user 101, 106, 110 to access the network while providing first name of the user 101, 106, 110 only to protect the privacy of the individual. The user 101, 106, 110 controls the amount of personal information they share instead of being forced by the network to reveal their first and last name. The network may require verification that the first name provided is the actual first name of the user 101, 106, 110 in order to verify the identity of the user 101, 106, 110 and protect members 103, 104, 108, 109 from scams or other harmful members 103, 104, 108, 109.

Groups 203, 204, 205 may be a collection of people with a hobby in common, such as a local fishing group, a national organization, such as the American Cancer Society, a school district, an entity, a church singles group, etc. Each group 203, 204, 205 may set up a group page containing information about the group 203, 204, 205 similar to a user's personal page. The group 203, 204, 205 page may be set up and facilitated by one administrative user or a set of administrative users 101, 106, 110. Similar to a user's 101, 106, 110 data information and personal page, groups 203, 204, 205 may supply data that is visible to connected user and connected members.

Each group 203, 204, 205 controls the privacy required to access the group's data page. Some groups 203, 204, 205 may allow an anonymous mode user 101, 106, 110 to access all of the features and information on their page while other groups 203, 204, 205 do not allow anonymous mode users 101, 106, 110 or anonymous mode members 103, 104, 108, 109 to view the details on their group data page. Each group 203, 204, 205 controls the privacy of their data age and the amount of information required to be disclosed by user 101, 106, 110 and members 103, 104, 108, 109 to access their group data page.

The data page of a user 101, 106, 110, member 103, 104, 108, 109 and groups 203, 204, 205 provides an organized online forum for sharing information and experiences with the like-minded sober member 103, 104, 108, 109 and group 203, 204, 205 in addition to the main page of the sober network 202. The data pages and group boards 204 may contain links to resources, articles, upcoming events, pictures, videos, questions, or any variety of information that supports others and themselves in supporting a sober lifestyle in a communication platform that supports sober living.

The concept of networking on this platform is to expand the user 101, 106, 110, groups 203, 204, 205, and members'103, 104, 108, 109 social network of sober contacts and providing information and access to sober events that encourages person-to-person contact. Remaining sober requires resources, guidance and experiences reinforcing sobriety that are limited on other social networks. Other networks facilitate setting goals and focusing content discussion with like-minded individuals but there is no current network or system that connects sober individuals so uniquely, effectively, and efficiently as depicted in the present invention. Furthermore, there are no prior art inventions that teach or suggest a method or system as illustrated in the present invention.

After successfully logging 200 into the network 100, the user 101, 106, 110, group administrator 203, 204, 205, or member 103, 104, 108, 109 is prompted with an optional client sober pledge or certification 201. This pledge 201 allows the user 101, 106, 110, group administrator 203, 204, 205, or member 103, 104, 108, 109 to certify 201 that they are sober on the present day and intend to remain sober on that day. Because sobriety is a challenge for an addict and this network focuses on being a resource for the addict, the optional client sober pledge or certification 201 provides the user an opportunity to pledge 201 their commitment to living a sober lifestyle. This pledge 201 provides an accountability to remaining sober that no other social media networks currently provide. The pledge 201 allows the user 101, 106, 110, group administrator 203, 204, 205, or member 103, 104, 108, 109 to focus on their sobriety before they view, post, comment, read other comments, sign up for events, or navigate through the network. The pledge 201 sets the tone for entering the sober network 100. The user 101, 106, 110, group administrator 203, 204, 205, or member 103, 104, 108, 109 can select to bypass this certification 201 and proceed to the network 100 without certifying they are sober 201 if they choose to bypass the certification. Access to the network 100 is allowed if the user 101, 106, 110, group administrator 203, 204, 205, or member 103, 104, 108, 109 bypasses the optional sober 201certification.

In FIG. 2, client and user 201 are interchangeable terms used to describe one person who logs into the network to access network features and resources. The optional client sober pledge 201 when bypassed does not block access to the site. If the user 101, 106, 110 chooses to certify 201 they are sober that day, the network will log that certification 201 and count the number of sober certifications 201 for the user 101, 106, 110 as a saved user activity. The user 101, 106, 110 can choose to add that sober certification 201 information and current count of sober certifications 201 to their personal data to show other members 103, 104, 108, 109 and groups 203, 204, 205 of their continuous sober status.

In one embodiment, the network 100 only logs the sober certification 201 on the user's 101, 106, 110 personal page containing personal information if the user 101, 106, 110 gives permission for the network to save. If the user 101, 106, 110 chooses to save this sober certification 201, then the counted saved user 101, 106, 110 certifications 201 are displayed as a on the user's 101, 106, 110 data page to show the number of consecutive sober days. If the user 101, 106, 110 misses a certification day, the user 101, 106, 110 can manually update the number of consecutive sober days. Or alternatively, if the user 101, 106, 110 relapses, the user 101, 106, 110 can reset the counter to begin a new count on their sober journey.

Due to a multitude of personal reasons, some users 101, 106, 110 o members 103, 104, 108, 109 may choose not to display any personal information but access the social network 100 without leaving any personal fingerprint. Anonymous login mode 200 allows the user 101, 106, 110 to login to the site with minimal personal information. While anonymous login mode 200 allows the user 101, 106, 110 to access the social network 100, it may not allow the user 101, 1 06, 110 to utilize all of the network functions.

An anonymous user 101, 106, 110 is required to login with a first name only and allowed access to view comments, activities or events, and group pages. This mode of network usage mirrors information an addict is usually required to divulge in recovery programs and meetings. For example, Alcohol Anonymous only requires the participant to divulge their first name at meetings and events. This allows the participant and user 101, 106, 110 in this invention to participate in the resource while maintaining their privacy.

Additionally, other social media networks and websites also require the user 101, 106, 110 to login first and last name for access to the network. For example, Facebook requires the user 101, 106, 110 to divulge both first and last name in order to access the network requiring the user 101, 106, 110 to divulge more information than the user 101, 106, 110 desires in order to view an event the user 101, 106, 110 desires to attend. The sober group in the present invention solves this privacy issue because the sober network does not require the user 101, 106, 110 to divulge any information except for their first name to view the event on the group 203, 204, 205 page and participate in the sober activity.

Traditional sober events organized by major rehabilitation resource organizations are intentionally set up so that participants'identities are kept strictly private. This level of privacy is important for a multitude of reasons including but not limited to protecting the privacy of participants by allowing participants to attend functions without having their true identity exposed to their family, friends, workplace, church or a variety of other groups or facets of the user's life. However, the sober social activity details about the event when posted on a private Facebook page that provide location and event details to all members of the sober group in a convenient online network, again requires the user to divulge both first and last name in order to access the network.

The purpose of the group 203, 204, 205 page in the present invention is to streamline and easily connect sober minded members focused on gaining and keeping their sobriety in a confidential manner when the user 101, 106, 110 prefers to divulge his first name only. While Facebook allows a streamlined and easy way to connect users 101, 106, 110 on a group 103, 104, 108, 109 page about upcoming events, it does not protect their identity with anyway to shield their last name from the group 103, 104, 108, 109 activity page or sober events hosted by a group 103, 104, 108, 109. The current invention solves this privacy problem by allowing the anonymous user 101, 106, 110 to access group 103, 104, 108, 109 pages, activities, and sober groups 103, 104, 108, 109 by only providing the user's 101, 106, 110 last name therein turn protecting their true identity from the group.

The anonymous user 101, 106, 110 in the present invention is also allowed to post comments, send or receive private messages, or sign up for activities and events without providing user's last name. After the user logs in to the network 200, opts to complete or skip the client/user sober pledge 201, then the user 101, 106, 110 may view the homepage of the network. The network is an online social platform that allows user 101, 106, 110 to create a profile with the amount of private information the user 101, 106, 110 is comfortable releasing to other members on the social network. Once the user creates a profile, the user can interact with members 103, 104, 108, 109 on the website. The user can also search group 103, 104, 108, 109 profiles, group 103, 104, 108, 109 activity pages, and network suggested groups 103, 104, 108, 109 for the user 101, 106, 110.

Groups 103, 104, 108, 109 may decide on their own privacy selection preference if they will allow anonymous users 101, 106, 110 that only provide their first names as login information 200 to participate in their group 103, 104, 108, 109 pages empowering the groups 103, 104, 108, 109 to control the amount of information required to access the site. The group 103, 104, 108, 109 itself sets the privacy requirement to join the group 103, 104, 108, 109; not the social network 100. Other social media networks and webpages set the privacy requirement and the user 101, 106, 110 and group 103, 104, 108, 109 must abide by their settings. In this present invention, privacy-setting choices are customizable by the user 101, 106, 110 and the group 103, 104, 108, 109. Empowering the group 103, 104, 108, 109 and user 101, 106, 110 to set privacy standards to their comfort and desired level of sharing.

Group 103, 104, 108, 109 pages may be any social, religious, school, school district, municipality, charity, or variety of other organizations that also support a sober lifestyle. While the social network 100 itself does not post any content on religion or politics, the users 101, 106, 110, members, and groups 103, 104, 108, 109 may post content on any subject with the restriction that it not encourage drug or alcohol use. For example, a municipality choose to post their events on this social network in order to foster and encourage sober lifestyle choice such as a city wide day of trash pick up to encourage citizens to take pride in their city. While the activity is not specifically centered on being sober or living a sober lifestyle, the activity will be free of alcohol and a great opportunity for the community to participate in an activity with others.

Another example, is a church singles group 103, 104, 108, 109 is going to host a dodgeball tournament. The church wants all members of the community to come out and participate in their dodgeball tournament. Because this is a sober event, the event will be posted to the calendar of the social network for all user and members to see. This again is a fun way for sober people to attend an event that is not focused on sobriety specifically but will be an event without alcohol or drugs present. Having a network site for sober events from a variety of sources with a variety of events connects all walks of life from the community that share a sober lifestyle focus.

Another example is a group 103, 104, 108, 109 of local sober friends are going to see a movie in their small town to see a popular new movie release. One member creates an event and invites ten of his member connections to join him at the movie. The movie may not be completely devoid of alcohol or drug references, but the group of members attending the movie will be sober. This event allows a group of individuals that do not belong to one group or organization to plan and attend a sober event with ease. The member that sets up the event can also set the privacy settings of the event so that other members can invite their member connections to join the movie event as well. Or the member can make the event a closed group and only invite the selected members. Either way, the group organizer can set the privacy settings of the event.

Private messages 205 are another way to communicate within the social network site. Because group pages 103, 104, 108, 109 and personal pages are viewable by the user's 101, 106, 110 connections or members 103, 104, 108, 109 of the group, a way to send a private message is through a private messaging chat system. Private conversations happen for a variety of different reasons and allow the user 101, 106, 110 to communicate in private rather than open for the other members to view. Depending on the subject and comfort level of the user 101, 106, 110, private messages allow the user 101, 106, 110 to freely communicate sensitive matters with ease on the social network 100.

Once the user 101, 106, 110 visits the desired pages, content pages, group 103, 104, 108, 109 pages, events page, and reviews any private messages, the user 101, 106, 110 then logs out of the social network. At any point in the user's 101, 106, 110 maneuvering on the social network site, the user 101, 106, 110 may log out of the network at any time or remain logged in if desired. There is no maximum or minimum usage to remain a current user on the social network site.

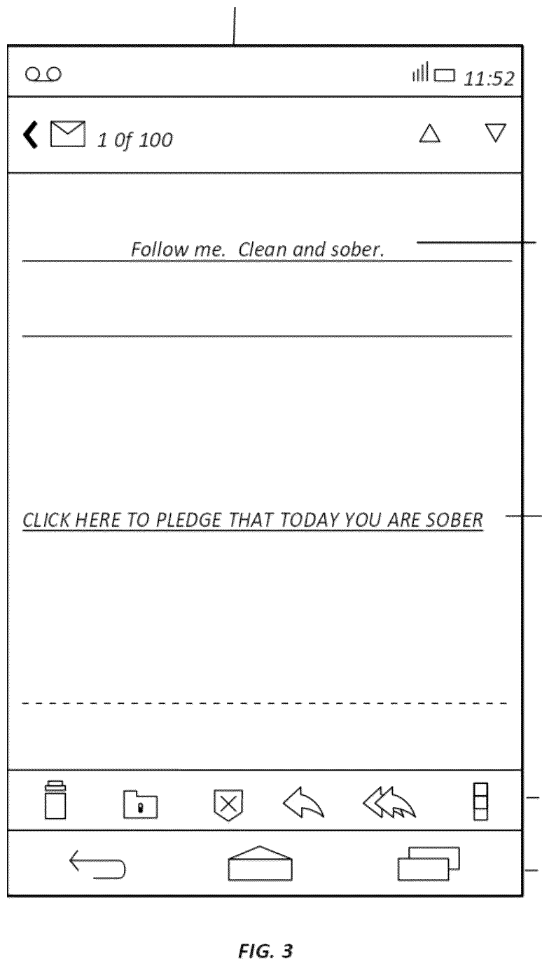

FIG. 3 illustrates an example network page 300 on a mobile device. The example page shows the optional certification or pledge that the user 101, 106, 110 may certify that day is sober 302 and the link that allows the user 101, 106, 110 to bypass the certification 302 and still access the social network site 301. The user 101, 106, 110 may click on the link that says “Click here to pledge that today you are sober” or the user may choose not to pledge that day that they are sober and click on the link that says “Follow me. Clean and sober.” Both links take you to the main page of the social network after the entry selection is made. The specific phrasing of the links to access the main page of the social network are used here as an example only and may be revised within the sprit and scope of the present invention.

If the user chooses the link to pledge that user 101, 106, 110 currently is sober and commits to remain sober the remainder of that day, the network logs that sober pledge selection and keeps a running count of the user's 101, 106, 110 pledges. The user 101, 106, 110 selects in his privacy settings whether he wants the number of pledges made by user 101, 106, 110 to be posted on his personal page. If he chooses to allow this content on his personal data page, then there will be a badge of sorts on his page showing the actual number of consecutive pledges the user 101, 106, 110 made declaring sobriety. The user 101, 106, 110 can also choose to take the sober pledge at login but not post that information on their personal page. The user 101, 106, 110 controls the posted data.

If the user chooses the link “Follow me. Clean and sober,” then the user 101, 106, 110 is taken directly to the homepage of the social network without logging any information for the user 101, 106, 110. The user 101, 106, 110 is allowed the full use of the social network without completing the sober pledge. The user's 101, 106, 110 access is only affected by the user's 101, 106, 110 decision to login in the anonymous mode. The user 101, 106, 110 can then maneuver around the site as the user 101, 106, 110 desires. The user's 101, 106, 110 personal page, group pages 103, 104, 108, 109, events and member personal pages are controllable by the icons on the bottom of the screen. Each icon 303, 304 links the user 101, 106, 110 to a different section or element of the social network site easily navigable by the user 101, 106, 110. The user 101, 106, 110 can create shortcuts as icons 303, 304 on the bottom of the home screen for easy access to those group pages 103, 104, 108, 109. Other customizable options are available for user 101, 106, 110 to customize the mobile application experience for the user 101, 106, 110.

FIG. 3 displays the ease of use of the social network on a mobile device 300. People of all different ages use their mobile device as a connection or lifeline to almost everything and everyone around them. The smart phone application 300 example as shown in FIG. 3 may be altered or improved from the example but the spirit and scope of the invention remains the same regardless of software updates, upgrades or tweaks.

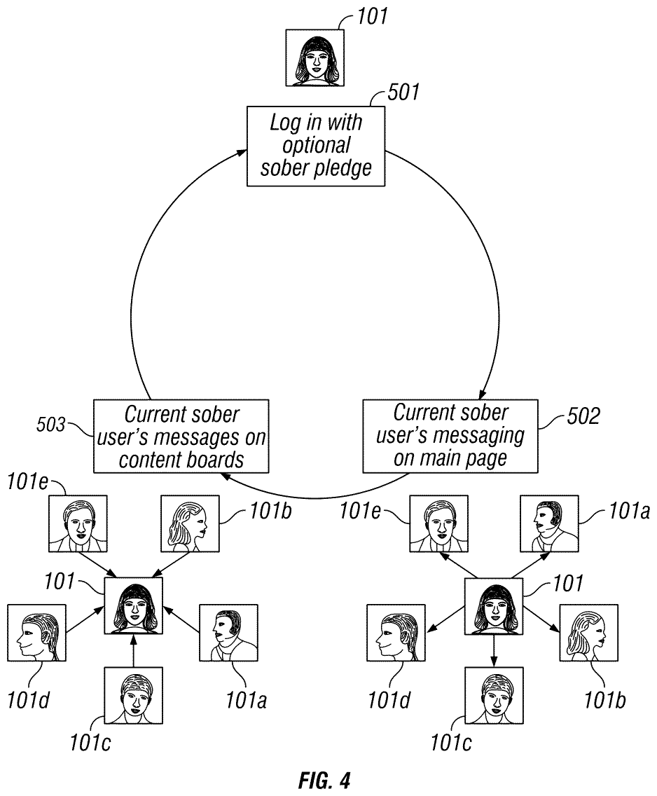

FIG. 4 illustrates an example sharing of sober content from a user 401 perspective. User 401 logs in to the sober social network in two mode options: full user mode or anonymous mode. User 401 makes a privacy choice when selecting which mode to choose as user's 401 log in method. The user 401 can set up their log in mode preference, as both modes are available for user 401 depending on the user's 401 preferred method. A user 401 can set up his login to only operate in full user mode, anonymous mode, or both so that the user 401 can choose the mode on any given day. As previously discussed, the user 401 has the option to choose her login mode and can alternate between the two modes as the user 401 chooses.

Immediately after logging in, the user 401 is prompted with the option to take the sober pledge 402. User 401 may choose to take the sober pledge 402 or user 401 may elect to bypass the sober pledge 402. If the user 401 selects to take the sober pledge 402, the user's 401 personal data will be updated with the current day's pledge 402. If the users 401 opts to not take the pledge 402, the user's 401 personal data will not be updated as there is no pledge 402 to update the user's 401 data. The user's 401 selection to take the pledge 402 or ignore the pledge 402 does not affect the content the user 401 is able to view on the network. After user 401 makes her selection of either taking the pledge 402 or bypassing the pledge 402, the user 401 is then redirected to the homepage of the network.

The network homepage 404 consists of sober content posted by members the user 401 connected with, advertisers who post sober-friendly content, groups, or any person or entity that is a member 412, 413, 415, 417, 418 of the sober social network. The user 401 scrolls through the posts similar to other social network sites. The user 401 may see posts from a variety members 412, 413, 415, 417, 418. These members 412, 413, 415, 417, 418 may be located near the user or hundreds perhaps thousands of miles away from the user 401. Location does not have any bearing on the ability to post on the site. The user 401 views the posts and comments of other members 412, 413, 415, 417, 418 they connected with on the netwrok. The user 401 may set up their personal data so that only members 412, 413, 415, 417, 418 they connected with can see their data or they can set their page as a public page meaning anyone on the site can view their posts and comments.

If the user 401 sets their privacy settings to a private page where only their member 412, 413, 415, 417, 418 connections may view their posts and comments, then the user 401 needs to connect with members 412, 413, 415, 417, 418 to allow their connected members 412, 413, 415, 417, 418 to view their posted content. User 401 or member 412, 413, 415, 417, 418 may send a request to members 412, 413, 415, 417, 418 to connect. The user 416 or member 412, 413, 415, 417, 418 receives a request by another member 412, 413, 415, 417, 418 to “connect” on the social network. If the user 416 or member 412, 413, 415, 417, 418 accepts the request to connect, then the user 416 and member 412, 413, 415, 417, 418 can see each other's posted content 404 and personal data page.

Other privacy settings may also be encompassed on the network so that the user 416 can select to allow the member's 412, 413, 415, 417, 418 they connected with to let the connection of a connection view their posts. This broadens the scope of who can view the user's 416 posts and comments allowing the user 416 to connect with more people. This method is more private than making every post or comment public because the user 416 is only allowing connections 412, 413, 415, 417, 418 or connections of the user's 416 connection to view their personal data and posts but allows the user 416 to potential make new connections through the user's 416 current connections. There is one degree of separation to the unknown member the user 416 chooses to share data with using this method instead of posting without any limitation on who can view the user's 416 data. The user 416 may also select the privacy setting on a public setting where anyone whether they are a connection to the user or not may view the user's data page or comments. Members and group pages also have the option to use a public privacy setting.

The content on the user's 416 messaging on the main page 404 consists of posts from either members, entities or member's connections based on the user's 416 privacy selection. The user 416 scrolls through posted comments, data, links to information, events, or any variety or combination thereof. The user 416 views comments from members about photos or any variety of information posted. The user 416 may also view comments from an administrator of a municipality page 418 concerning an upcoming event that the user 416 is interested in attending.

The user 416 may also view a comment from a member's 417 connection the user 416 has not yet connected with if the privacy setting selected by member 417 and user 416 allows the user 416 to view the member's 417 connections regardless of whether the member's 417 connections have connected with the user 416. This privacy setting allows friends of friends to be visible to the user 416. The member's 417 connection must also select the same privacy setting to be visible to the user 416 if the user 416 is not connected to said connection. When user 416 and member 417 select privacy settings that allow connections of connections'comments and posts to be visible to one degree of separation connection, then the potential connections and reviewable content for user 416 is greatly expanded by this privacy setting. These example privacy settings and comments are included for reference only and are not a complete definitive list of settings available on the network.

In the current example of the user's 416 sharing and viewing of content on the main page 404, the user 416 may see a post that the user 416 wants to review further. For example, a post from an administrator of a municipality page 418 details an upcoming event the user 416 may be interested in attending. The user 416 may click on the municipality's content page 405 to see additional information about the event. The municipality's content page 405 contains the date, time, location, and specifics concerning the upcoming event but user 408 has a question about the location. The user 408 may post a question on the municipality's content page 405 to obtain an answer to her question. Depending on the municipality's content page 405 privacy settings, the user 408 may receive answers to her questions from other members 407, 411, 410, 409 or directly from the administrator of the content page 406.

The members 407, 411, 410, 409 of the content page may also post their own questions to the content page 406 that user 408 can view and post an answer directly on the municipality's content page 405 if she so chooses. The user 408 could then connect with members 407, 411, 410, 409 of the municipality's page 405 if she had not connected with them previously. The municipality's page 405, the user 408 commented on, is located in the user's 408 current city of residence so the user 408 is interested in making additional sober connections online with this particular group 405 in addition to the possibility of attending an event in person. The sober social network allows the user 408 an opportunity to connect online so that network connection develops into user 408 attending events, gatherings, or meetings in person with like-minded sober members 407, 411, 410, 409.

Another example user 401 is a young college female who was recently released from a rehabilitation facility returning to her hometown. Because user 401 has been away at college for two years along with most of her friends, user 401 is interested in connecting with other college age kids who are sober in user's 401 hometown. Before going to rehab, user 401 would meet new people at bars or social events that centered around drinking as most college aged adults do. Since user 401 is now sober, user 401 needs a fresh way to meet people that are supportive of her new sober lifestyle. Since getting out of the rehabilitation facility, user 401 has not been able to participate in typical college activities because of the temptation to drink at social functions and has not been able to expand her circle of friends to include sober friends.

This example user 401 logs into the social network, takes the sober pledge 402, views the main page 404 of the network, and quickly makes a few connections to members 417, 415, 413, 412, 418 she knows through her sobriety program that are also on the social network. When user 416 connects with member 415, she sees that member 415 is connected with a local group of college age kids that also adhere to a sober lifestyle. User 416 clicks on the content boar 405 for the local group 405 of college adults to see if user 416 can make any additional sober connections. User 408 clicks on one of the potential new connection's personal data page 410 and requests a connection 410 to a similar aged college adult.

The user 408 also sees that the potential new connection 410 is connected to a college church ministry who also has a content page 405 that frequently holds events for college kids. User 408 enjoys bowling as an activity and sees an event posted on their data page that the college church ministry group 405 has a bowling event coming up in the next few weeks. User 408 clicks on the event page for the bowling event to review the specifics about the event. User 408 then signs up to attend the upcoming event knowing that the event will be a sober event.

Prior to logging on to the sober social network and making new sober connections, the user 408 was very isolated and unhappy due to her limited social options and few sober friends that was leading her to a possibility of relapsing. However, after creating a personal data page profile, connecting with members, connecting with groups, and attending sober events, the user 408 is making great progress in her commitment to remaining sober through the resources and connections user 408 made on the sober network.

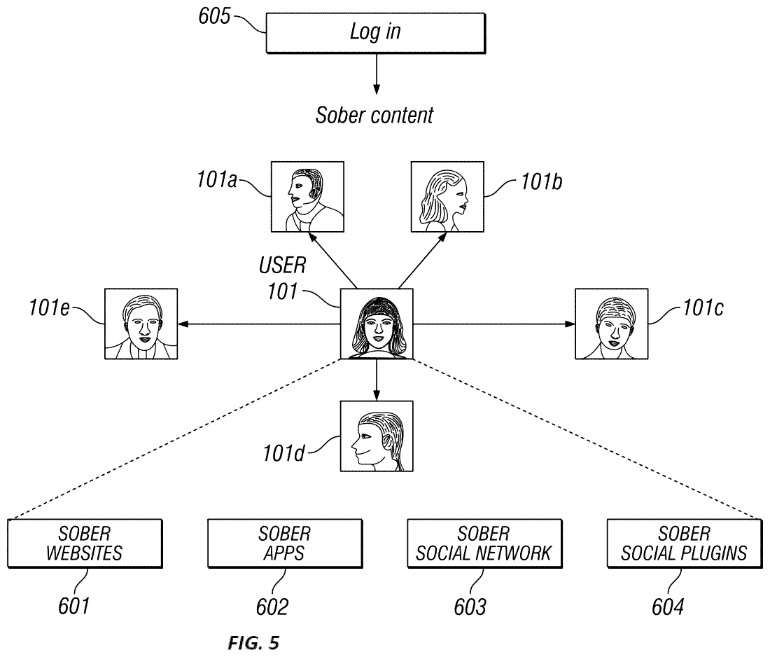

FIG. 5 is illustrates an example social graph for the sober social network. FIG. 5 shows one example of the user's 501 exposure to different members 505, 502, 503, 504, group pages 509, sober events, website links 507, advertisements 508, plugins 510 and potential connections. The entire network FIG. 5 is a sober resource for the user 501 and members 505, 502, 503, 504. The number and scope of sober connections is limitless. After user 501 logs 500 into t he network, the user 501 connects with members 505, 502, 503, 504 the user 501 already knows from current and past experiences that leads the user 501 to new connections 506 user 501 more than likely never would met or cross paths but for this sober network. Those new connections 506 lead to a group page 509 with multiple resources now available to the sober user 506 including but not limited to sober websites 507, sober advertisements 508, sober social network of local events linked on sober group pages 509, and sober social plugins 510. This example on FIG. 5 is only an illustration of potential connections and does not capture the extent or limit of connections available on the social network for the user 501, members 505, 502, 503, 504, 506 or groups 509.

It should be noted and understood that various changes and modifications to the described preferred embodiments herein will be evident to those skilled in the art. Such changes and modifications can be made without deserting from the spirit and scope of the present invention and without weakening its intended advantages. It is therefore intended that all or any such changes and modifications be enclosed by the appended claims.

Any element in a claim that does not explicitly state “means for” performing a specified function, or “step for” performing a specific function, is not to be interpreted as a “means” or “step” clause as specified in 35 U.S.C. § 112, ¶ 6. In particular, the use of “step of” in the claims herein is not intended to invoke the provisions of 35 U.S.C. § 112, ¶ 6.

We claim:

1. A method for sober based social networking comprising the steps of:
receiving, from a user, a login information into a network platform using a personal computing device, wherein the network platform is configured to select automatically layouts fit to accommodate different screen resolutions and applications designed to work specifically on different computing devices;
registering the user on the network platform in a full user mode and an anonymous mode to provide the user an option to choose from the full user mode and the anonymous mode and alternate between the full user mode and the anonymous mode, wherein accessibility to the network platform is provided in one of said modes based on said login information;
receiving a query from the registered user regarding rehabilitation;
generating a list of one or more medical practitioners registered with the network platform based on a processing of the query; and
creating communication links with the one or more registered medical practitioners based on a selection by the registered user, wherein the registered user communicates with the one or more registered medical practitioners in one of an anonymous mode and a full mode depending upon a stage of communication.

2. The method according to claim 1, wherein the list of network platform registered medical practitioners is verified from a government database of registered medical practitioners.

3. The method according to claim 1, wherein the selection from the list of network platform registered medical practitioners is based upon a response to anonymous user queries related at least to an approach, treatment methods, and success rate.

4. The method according to claim 3, wherein the anonymous user queries is selected from a set of predefined questions, or custom queries.

5. A system for sober based social networking, comprising:
a personal computing device, configured to:
receive login information from a user;
a network platform, operatively coupled to the personal computing device, configured to:
register the user in a full user mode and an anonymous mode, providing the user an option to choose from the full user mode and the anonymous mode and alternate between the full user mode and the anonymous mode, wherein accessibility to the network platform is provided in one of said modes based on said login information, wherein the network platform is configured to automatically adjust layouts to accommodate different screen resolutions and applications designed to work specifically on different computing devices;
receive a query from the registered user regarding rehabilitation;
generate a list of one or more medical practitioners registered with the network platform based on a processing of the query; and
create communication links with one or more registered medical practitioners based on a selection by the registered user, wherein the registered user communicates with the one or more registered medical practitioners in one of an anonymous mode and a full mode depending upon a stage of communication;
a database, operatively coupled to the network platform, configured to:
store user information, including login credentials, user mode preferences, and query history;
store the generated list of one or more registered medical practitioners, including the one or more medical practitioners'contact details and areas of expertise;
a server comprising a processor memory, and a communication interface operatively coupled to the network platform and the database, configured to:
process the user's query regarding rehabilitation;
match the query with the areas of expertise of one or more medical practitioners registered in the database;
generate and provide the list of matched medical practitioners to the user for selecting a medical practitioner.
